SEQUENCE LISTING (5 sequences)

SEQ ID NO: 1 - DNA, 1866 bp, Adeno-associated virus serotype 2
  source 1..1866: mol_type = genomic DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaatag

SEQ ID NO: 2 - DNA, 208 bp, Adeno-associated virus serotype 2
  source 1..208: mol_type = genomic DNA
gtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggt

SEQ ID NO: 3 - DNA, 19 bp, synthetic construct
  source 1..19: mol_type = other DNA; note = TetR binding site
tccctatcagtgatagaga

SEQ ID NO: 4 - DNA, 107 bp, synthetic construct
  source 1..107: mol_type = other DNA; note = AV modified MLP sequence
cgccctcttcggcatcaaggaaggtgattggtttgtaggtgtaggccacgtgaccgggtgttcctgaaggggggctataaaaggtccctatcagtgatagagactca

SEQ ID NO: 5 - DNA, 107 bp, synthetic construct
  source 1..107: mol_type = other DNA; note = AV modified MLP sequence
cgccctcttcggcatcaaggaaggtgattggtttgtaggtgtaggccacgtgactccctatcagtgatagagaactataaaaggtccctatcagtgatagagactca

(19) United States
(12) Patent Application Publication
(10) Pub. No.: US 2023/0257831 A1
(43) Pub. Date: Aug. 17, 2023

(54) METHOD FOR DETERMINING AAV TITRE

(71) Applicant: Oxford Genetics Limited, Oxford, Oxfordshire (GB)

(72) Inventors: Ryan Cawood, Oxford (GB); Weiheng Su, Oxford (GB)

(21) Appl. No.: 17/823,680

(22) Filed: Aug. 31, 2022

Related U.S. Application Data
(60) Provisional application No. 63/239,789, filed on Sep. 1, 2021.

(51) Int. Cl. C12Q 1/70

(52) U.S. Cl. CPC C12Q 1/701; C12Q 1/6806

(57) ABSTRACT

Methods of determining the titre of recombinant or wild-type adeno-associated viruses (AAVs) in a sample of recombinant or wild-type AAVs, respectively, are provided. The methods utilise recombinant adenoviruses to amplify the number of AAVs. In some embodiments, the genome of each recombinant adenovirus comprises a rep gene. In some embodiments, the genome of each recombinant adenovirus comprises a repressor element in the Major Late Promoter (MLP).

### CROSS-REFERENCE

This application claims the benefit of priority from U.S. Provisional Application No. 63/239,789, filed Sep. 1, 2021, which is incorporated by reference in its entirety.

### FIELD OF THE INVENTION

The present invention relates to methods of determining the titre of recombinant or wild-type adeno-associated viruses (AAVs) in a sample of recombinant or wild-type AAVs, respectively. The methods utilise recombinant adenoviruses to amplify the number of AAVs. In some embodiments, the genome of each recombinant adenovirus comprises a rep gene. In some embodiments, the genome of each recombinant adenovirus comprises a repressor element in the Major Late Promoter (MLP).

### SEQUENCE LISTING STATEMENT

A computer readable form of the Sequence Listing is filed with this application by electronic submission and is incorporated into this application by reference in its entirety. The Sequence Listing is contained in the file created on Aug. 29, 2022 having the file name “21-1144-US_Sequence-Listing_ST26.xml” and is 8 kb in size.

### BACKGROUND OF THE INVENTION

For many applications, it is necessary to determine the viral titre (i.e. the number of infectious viral particles per unit volume) of a stock of wild-type (wt) AAV particles or recombinant AAV (rAAV) particles.

In general, a small portion of the virus particle stock solution is used to infect a population of target tissue culture cells; the cells are incubated and then examined for a biomarker; and the viral titre is calculated based on the proportion of cells that have that biomarker.

Infectivity of rAAVs can be measured by examining the cells or culture medium for the expression of a gene of interest (i.e. a transgene, such as EGFP, RFP, beta-galactosidase or luciferase, or antibody staining for the protein of interest); and the infectious titre of the virus preparation may be determined by the proportion of cells which are positive for expression of the transgene. A significant disadvantage of these methods is the lengthy time-period (approximately 7-10 days) required for accumulation of sufficient protein of interest (such as EGFP) to enable sensitive and reliable detection of infected cells.

In order to improve the sensitivity of these assays, the titration assay usually includes an amplification step, wherein the number of AAV particles is amplified, e.g. using adenoviruses.

A number of specific forms of titration assays are already known:

The replication centre assay (Clement and Grieger, 2016, “Manufacturing of recombinant adeno-associated viral vectors for clinical trials”, Mol. Ther. Methods Clin. Dev., 3: 16002) employs wild-type adenovirus and wild-type AAVs to supply essential genes for inducing replication of the rAAV vectors. The subsequent detection by qPCR and DNA probes has also been reported. (See also Francois, A. et al., 2018, “Accurate Titration of Infectious AAV Particles Requires Measurement of Biologically Active Vector Genomes and Suitable Controls”, Mol. Ther. Methods Clin. Dev. 10: 223-36). However, rAAV vector titration by this method is generally compounded by a lack of reproducibility due to the required need to balance the wild-type adenoviruses and wild-type AAVs.

AAV genes are generally deleted in rAAVs. Therefore, for the assay of rAAV particles which do not include rep genes, HeLa-based AAV packaging cell lines (such as HeLa RC32 cells, which stably encode the AAV rep and cap genes) are commonly used (e.g. Zen et al., 2004, “Infectious titer assay for adeno-associated virus vectors with sensitivity sufficient to detect single infectious events”, Hum. Gene Ther. 15: 709-15). In these assays, HeLa RC32 cells are infected with the recombinant AAV sample preparations and wild-type adenovirus to induce expression of the AAV rep and cap genes from the stable packaging cells. AAV infected cells are generally determined by detection of the reporter transgene (EGFP, RFP, beta-galactosidase or luciferase) or replication of the AAV transfer genome within infected cells using qPCR or Southern blotting assays. While these methods enhance the sensitivity for detection of AAV infected cells and shorten the time for quantification (approximately 2-3 days), they are limited to the use of rAAVs with AAV capsid serotypes which are capable of infecting these stable packaging cells.

An rAAV titration assay utilising a replication-defective Herpes simplex virus (HSV) vector expressing AAV rep and cap genes has also been described (Mohiuddin, I. et al., 2005, “Herpesvirus-based infectious titering of recombinant adeno-associated viral vectors”, Mol. Ther. 11: 320-6). However, the utility of this assay is limited to cells which are permissive to HSV.

Alternative approaches to determining the infectious titre of rAAV stocks using a DNA synthesis inhibitor and a chemical agent that increases the activity of the CMV promoter have been described (e.g. US 6,841,357). In this approach, recombinant AAV particles, encoding a reporter transgene under transcriptional control of the CMV promoter, are used for infection of target cells. Cells are treated with a DNA synthesis inhibitor and an agent that increases the activity of the CMV immediate early promoter to enhance expression of the transgene reporter for sensitive detection of infected cells.

This approach is limited, however, to the quantification of rAAV vectors encoding a reporter transgene (such as EGFP) and specific promoters (such as CMV), and wherein the transgene activity is increased by the DNA synthesis inhibitor and chemical agent.

There remains a need, therefore, for alternative methods for assaying AAV viral titres which are less restricted in the use of rAAVs with specific AAV capsid serotypes, which are not specific to the need for particular promoters or transgenes or host cells, and which do not require the additional presence of both wild-type adenoviruses and wild-type AAVs.

### SUMMARY OF THE INVENTION

In one embodiment, the invention relates to a method of determining the titre of rAAVs wherein the method comprises the use of a recombinant AV vector which comprises a rep gene. This obviates the need to provide the rep gene via the addition of wild-type AAVs, in the cell line or in an additional HSV vector. This recombinant AV vector may optionally also comprise a repressor element in the Major Late Promoter (MLP).

WO2019/020992 discloses that the transcription of the Late adenoviral genes can be regulated (e.g. inhibited) by the insertion of a repressor element into the Major Late Promoter. By “switching off” expression of the adenoviral Late genes, the cell’s protein-manufacturing capabilities can be diverted toward the production of a desired recombinant protein or AAV particles. This system is known as a TERA (or TESSA) system.

The inventors have had the insight to realise that the use of this TERA system in the context of an AAV titration assay would provide a number of advantages.

First, the use of a TERA adenoviral vector provides adenovirus helper genes to enable AAV replication, but it avoids the production of compounding adenovirus particles. This makes it easier to detect the rAAV. Second, the use of this TERA system avoids the health risks associated with using infectious adenoviruses. Third, cells which are infected with wild-type adenoviruses undergo cell lysis at 2-3 days after infection, due to production of adenovirus particles, therefore limiting the timeframe for reporter analysis. The use of the TERA system avoids the production of adenovirus particles and cell lysis, which enables a more reliable detection of the reporter transgene in infected cells for rAAV quantification. Fourth, the use of a TERA adenoviral vector with a rep gene, but retaining an intact adenoviral E1 region, means that the method may be carried out in any permissive cell type, i.e. the method is not limited to cell lines which express rep; for example, primary cells may be used.

In another embodiment, the invention relates to a method of determining the titre of wild-type AAVs, which comprises the use of a recombinant AV vector which comprises a repressor element in the Major Late Promoter (MLP). This embodiment also benefits from the advantages discussed above. In this embodiment, the rep gene is provided in the wild-type AAVs and hence it is not necessary to provide the rep gene in the recombinant adenovirus.

In one embodiment, the invention provides a method of determining the titre of recombinant adeno-associated viruses (rAAVs) in a sample of rAAVs, the method comprising the steps of:
(a) providing each of components (i)-(iii) in one or each of a set of discrete compartments:
   (i) a population of host cells;
   (ii) a population of recombinant adenoviruses, wherein the genome of each recombinant adenovirus comprises a rep gene; and
   (iii) a solution having a defined level of dilution of the sample of rAAVs,
   wherein the host cells are ones which are capable of being infected by the recombinant adenoviruses and by the rAAVs;
(b) culturing the discrete compartments under conditions such that the host cells are infected by the recombinant adenoviruses and rAAVs; and
(c) determining the level of a biomarker for each of the discrete compartments, wherein the biomarker is one which is representative of the number of rAAVs, and thereby determining the titre of the rAAVs in the sample.

In a preferred embodiment, the genome of each recombinant adenovirus additionally comprises a repressor element in the Major Late Promoter (MLP).

In another embodiment, the invention provides a method of determining the titre of wild-type adeno-associated viruses (AAVs) in a sample of AAVs, the method comprising the steps of:
(a) providing each of components (i)-(iii) in one or each of a set of discrete compartments:
   (i) a population of host cells;
   (ii) a population of recombinant adenoviruses, wherein the genome of each recombinant adenovirus comprises a repressor element in the Major Late Promoter (MLP); and
   (iii) a solution having a defined level of dilution of the sample of AAVs,
   wherein the host cells are ones which are capable of being infected by the recombinant adenoviruses and by the AAVs;
(b) culturing the discrete compartments under conditions such that the host cells are infected by the recombinant adenoviruses and AAVs; and
(c) determining the level of a biomarker for each of the discrete compartments, wherein the biomarker is one which is representative of the number of AAVs, and thereby determining the titre of the AAVs in the sample.

This embodiment of the invention is also applicable to rAAVs, mutatis mutandis, wherein the rAAVs comprise a functional rep gene in the rAAV genome.

In a preferred embodiment, in Step (a) component (iii) is provided (e.g. dispensed) into different subsets of compartments within the set of discrete compartments, wherein different subsets of compartments receive different defined levels of dilution of the sample of AAVs, and Step (c) comprises (c) determining the level of a biomarker for each of the subsets of discrete compartments, wherein the biomarker is one which is representative of the number of AAVs, and thereby determining the titre of the AAVs in the sample.

Preferably, in Step (a), a first subset of compartments (e.g. rows of a micro-titre plate) receive a first defined level of dilution of the sample of AAVs, there are n subsets of compartments (e.g. n rows), and the nth subset of compartments receives a 10⁻⁽ⁿ⁻¹⁾ dilution of the first defined level of dilution of the sample of AAVs, wherein n=2 to 20.

The invention also provides a method of determining the TCID₅₀ (Median Tissue Culture Infectious Dose) of a population of wild-type or recombinant AAVs, the method comprising the steps:
(a) performing a method of the invention, wherein different subsets of compartments (e.g. rows in a micro-titre plate) receive different defined levels of the sample of AAVs and the different defined levels vary by the serial dilution factor, d; and
(b) calculating the TCID₅₀ using the formula:
$$T i t r e \left(T C I D 50 / m L\right) = \frac{{10}^{1 + D \left({X}_{0} + S - 0.5\right)}}{v o l u m e p e r c o m p a r t m e n t \left[m L\right]}$$
wherein
$$\text{D=log} \left(\text{d}\right) ;$$
X₀ = -log [the highest dilution factor at which the presence of AAVs has been detected in all compartments (e.g. wells) in a particular subset of compartments (e.g. rows in a micro-titre plate)]; and wherein, in the subsets of compartments (e.g. rows of a micro-titre plate) in which the presence of AAVs has not been detected in all compartments (e.g. wells of the micro-titre plate) in that subset, S is the sum of the ratios of the number of compartments (e.g. wells) in which AAVs have been detected to the total number of compartments (e.g. wells) in that subset of compartments (e.g. rows).

### BRIEF DESCRIPTION OF THE DRAWINGS

FIG. 1. Example of a Dilution Plate (96 well) layout for use with the method of the invention.

FIG. 2. Quantification of infectious titre of rAAV2-EGFP stocks, derived from TERA2.0 or helper-free plasmid transfection method, in HEK293 cells using TERA-RepCap adenovirus.

FIG. 3. DNA replication of rAAV-EGFP in A549 cells using TERA-E1-Rep virus, wherein the rep gene is integrated alongside the E1 genes within the recombinant adenovirus.

FIG. 4. Detection of rAAV2-EGFP infection events in HEK293 (using TESSA-E1-Rep) and HelaRC32 (using Ad-E1) cells by fluorescence microscopy (E), automated fluorescence cell imager (C), or cellular DNA extracted from each well and quantified by EGFP-specific qPCR (Q).

FIG. 5. TCID50 infectious titration of rAAV2-EGFP stock in HEK293 cells (using TESSA-E1-Rep) and HeLaRC32 (using Ad5-E1) cells determined using a fluorescence microscope (EVOS), automated fluorescence cell imager (Cell imager; CELLAVISTA) or total DNA was extracted from each well and measured by EGFP-specific qPCR.

FIG. 6. Infectious titration of rAAV2 in different cell lines with TERA-E1-Rep compared to HeLaRC32 method using the TCID50 assay.

FIG. 7. Genome copies to TCID50 ratio of AAV2-EGFP stock in different cell lines.

FIG. 8. Infectious titre of rAAV serotypes determined in HEK293 and A549 cells with TERA-E1-Rep using the TCID50 assay.

### DETAILED DESCRIPTION OF THE INVENTION

The invention provides methods of determining the titre of wild-type or recombinant adeno-associated viruses (rAAVs) in a sample of wild-type or recombinant AAVs, respectively. The term “titre” relates to the number of infectious AAV or rAAV particles per unit volume. Thus the term “title” may refer to “infectious titre”. The term “titre” may also refer to the “transduction titre”, wherein transduction refers to an AAV particle gaining entry into a cell and being capable of expressing its transgene in the cell.

The sample of wild-type or recombinant AAVs whose titre is being determined will generally be in the form of a liquid (e.g. aqueous) composition.

In Step (a), each of components (i)-(iii) is provided in (e.g. dispensed into) each of one or more discrete compartment or different subsets of discrete compartments. The compartments may, for example, be organised in a matrix or an array. The discrete compartments may, for example, be wells in a multi-well plate or micro-titre plate. The wells are for retaining components (i)-(iii) in isolation from other compartments.

In one embodiment, Step (a) comprises dispensing each of components (i)-(iii) into each of a set of wells or subset of wells in a multi-well plate.

Preferably, the discrete compartments are the wells in a 48-well, 96-well or 385-well culture plate. Each compartment receives the same volume of each components. The subsets of compartments may, for example, be specific rows or columns of a multi-well plate. The set of discrete compartments will comprise at least 2 subsets, preferably 3, 4, 5, 6, 7, 8, 9 or 10 or more subsets. Some of the compartments/wells in the multi-well plate or micro-titre plate may be left empty or comprise control reagents.

The population of host cells are cells which are capable of being infected by both the recombinant adenoviruses and the (wild-type or recombinant) AAVs, i.e. they are permissive for infection by the recombinant adenoviruses and the AAVs. It is necessary for both recombinant adenoviruses and AAVs to be present within the host cells in order for replication of the AAVs to occur. The host cells must be capable of being cultured in vitro.

The host cells are preferably at an adequate level of confluency, e.g. 85-95%, more preferably about 90% confluency, at the point at which the recombinant adenoviruses and AAVs are added. Confluency may be determined using a light microscope.

The host cells are provided in one or a plurality of discrete compartments (e.g. wells). Preferably, each discrete compartment comprises 5,000-50,000 cells, more preferably 10,000-30,000 cells, and most preferably about 20,000 cells.

Generally, the cells in the population of host cells will all be of the same type (i.e. species), although defined mixtures of different types of cells may be used.

Each of the discrete compartments receives the same cells and (essentially) the same number of cells.

The host cells are isolated cells, e.g. they are not situated in a living animal or mammal. The cells may be primary or immortalised cells (e.g. cell lines).

Preferably, the host cells are mammalian cells. Examples of mammalian cells include those from any organ or tissue from humans, mice, rats, hamsters, monkeys, rabbits, donkeys, horses, sheep, cows and apes. Preferably, the host cells are human cells.

Examples of human primary cells include BJ Fibroblasts, BJ hTERT Fibroblasts, ES cells, HUVEC cells, Karatinocytes and Hematopoietic Progenitor cells.

Examples of human cell lines include CaCo-2, HBEC, HEK 293, HeLa, HepG2, HT29, Jurkat, K562, MCF-7, TF1 a, Saos-2 and U20S cells.

Examples of mouse primary cells include Adult Skin Fibroblast, Astrocytes, ES cells, Hematopoietic Progenitor, Keratinocytes, Lung Epithelial, Lung Mesenchymal, Mesenchymal Stem, Embryonic Fibroblast, Skeletal Muscle Progenitor and White Adipose Progenitor cells. Examples of mouse cell lines include 3T3, C2C12 and MIN6 cell lines. Other cell lines includes CHO and COS-7 cells.

Preferred cells are A549, HeLa, HepG2, Cos-7, CHO, Astrocyte, ES cells, Fibroblast, HUVEC, Keratinocyte, HBEC, CaCo-2, HBEC, Jurkat, K562, U2OS, Huh7 and MCF-7 cells.

In some embodiments, the cells will have one or more adenoviral Early genes (e.g. E1A and/or E1B) stably integrated into the cell genome or present in an episome within the cell. This obviates the need for these proteins to be supplied on a Helper Plasmid or within the recombinant adenovirus.

Examples of such cells which have adenoviral E1A and E1B genes stably integrated into the host cell genomes include HEK293, PerC6 or 911 cells.

In other embodiments, the cells are ones which do not have one or more adenoviral Early genes (e.g. E1A or E1B) stably integrated into the cell genome or present in an episome within the cell.

Step (a) includes the step of providing a population of recombinant adenoviruses into one or each of the set of discrete compartments. Each of the discrete compartments receives the same recombinant adenovirus and (essentially) the same number of recombinant adenoviruses.

AAVs cannot replicate on their own; they require the presence of a helper virus, such as an adenovirus. In the presence of such a helper virus, AAV gene expression is activated, allowing the AAV to replicate using the host cell’s polymerase. The same is true for rAAVs.

As used herein, the term “recombinant adenovirus” refers to an adenovirus wherein one or more heterologous genes have been inserted or one or more other non-natural modifications have been made. The recombinant adenovirus does not therefore have a wild-type adenovirus nucleotide sequence. The recombinant adenoviruses must be capable of infecting the host cells. The recombinant adenoviruses provide sufficient helper genes to enable the AAV to replicate. Such helper genes include E1A, E1B, Adenovirus E4Orf6, the Adenovirus DNA binding protein (DBP), and the Adenovirus VA RNAs.

In one embodiment of the invention, each recombinant adenovirus comprises a rep gene inserted into the genome of the adenovirus. The rep may be integrated into the genome of the recombinant adenovirus at any suitable position, such that the recombinant adenovirus is still capable of providing sufficient helper functions and Rep polypeptides for the replication of the AAV. For example, the rep gene may be integrated into the genome of the recombinant adenovirus in the E1 region, E3 region or L5 region. In some embodiments, the rep gene is integrated within the E1 region of the recombinant adenovirus and the adenoviral E1 genes are intact. In other embodiments, the rep gene is integrated within the E1 region of the recombinant adenovirus and the adenoviral E1 genes are not intact. In such embodiments, the E1 helper gene products need to be provided by the host cell. Preferably, the rep gene is integrated with the E1 region of the recombinant adenovirus.

As used herein, the term “rep gene” refers to a gene that encodes one or more open reading frames (ORFs), wherein each of said ORFs encodes a Rep (preferably an AAV Rep) non-structural protein, or variant or derivative thereof. These Rep non-structural proteins (or variants or derivatives thereof) are involved in AAV genome replication and/or AAV genome packaging.

The wild-type AAV rep gene comprises three promoters: p5, p19 and p40.

Two overlapping messenger ribonucleic acids (mRNAs) of different lengths can be produced from p5 and from p19. Each of these mRNAs contains an intron which can be either spliced out or not using a single splice donor site and two different splice acceptor sites. Thus, six different mRNAs can be formed, of which only four are functional. The two mRNAs that fail to remove the intron (one transcribed from p5 and one from p19) read through to a shared poly-adenylation terminator sequence and encode Rep78 and Rep52, respectively. Removal of the intron and use of the 5’-most splice acceptor site does not result in production of any functional Rep protein - it cannot produce the correct Rep68 or Rep40 proteins as the frame of the remainder of the sequence is shifted, and it will also not produce the correct C-terminus of Rep78 or Rep52 because their terminator is spliced out. Conversely, removal of the intron and use of the 3’ splice acceptor will include the correct C-terminus for Rep68 and Rep40, whilst splicing out the terminator of Rep78 and Rep52. Hence the only functional splicing either avoids splicing out the intron altogether (producing Rep78 and Rep52) or uses the 3’ splice acceptor (to produce Rep68 and Rep40). Consequently, four different functional Rep proteins with overlapping sequences can be synthesized from these promoters.

In the wild-type AAV rep gene, the p40 promoter is located at the 3’ end. Transcription of the Cap proteins (VP1, VP2 and VP3) is initiated from this promoter in the wild-type AAV genome.

The four wild-type AAV Rep proteins are Rep78, Rep68, Rep52 and Rep40. Hence the wild-type AAV rep gene is one which encodes the four Rep proteins Rep78, Rep68, Rep52 and Rep40.

As used herein, the term “rep gene” includes wild-type AAV rep genes, and derivatives thereof and artificial rep genes which have equivalent functions to wild-type AAV rep genes. In one embodiment, the rep gene encodes functional Rep78, Rep68, Rep52 and Rep40 polypeptides. In another embodiment, the rep gene encodes functional Rep 78 and Rep 68 polypeptides. In some embodiments, the rep gene p19 promoter is non-functional.

The rep gene is preferably a viral gene or derived from a viral gene. More preferably, the rep gene is an AAV gene or derived from an AAV gene. In some embodiments, the AAV is an Adeno-associated dependoparvovirus A. In other embodiments, the AAV is an Adeno-associated dependoparvovirus B.

11 different AAV serotypes are known. All of the known serotypes can infect cells from multiple diverse tissue types. Tissue specificity is determined by the capsid serotype.

The AAV may be from serotype 1, 2, 3, 4, 5, 6, 7, 8, 9, 10 or 11. Preferably, the AAV is serotype 1, 2, 5, 6, 7, 8 or 9. Most preferably, the AAV serotype is 5 (i.e. AAV5).

It is recognised by those in the art that the rep genes of AAV vary by clade and isolate. The sequences of these genes from all such clades and isolates are encompassed herein, as well as derivatives thereof.

Preferably, the recombinant adenovirus also has one or more or all of the following features:
(i) there are no functional Rep p5 or p40 promoters;
(ii) no Rep adenovirus inhibitor sequence is present;
(iii) the Rep p19 promoter has been modified to delete the TATA box, although the promoter is still functional;
(iv) the rep gene is inserted in the E1 region of the adenovirus; and
(v) the transcriptional orientation of a heterologous promoter does not drive expression towards the Rep coding sequence.

The recombinant adenovirus is preferably provided at an MOI of 1-1000 infectious helper virus per cell.

Preferably, the recombinant adenovirus is an Ad 5 serotype.

The wild-type AAV (serotype 2) rep gene nucleotide sequence is given in SEQ ID NO: 1 In some embodiments, the term “rep gene” refers to a nucleotide sequence having at least 70%, 80%, 85%, 90%, 95%, 99% or 100% sequence identity to SEQ ID NO: 1 and which encodes one or more Rep78, Rep68, Rep52 and Rep40 polypeptides. In some embodiments, the term “AAV rep gene” refers to a nucleotide sequence having at least 70%, 80%, 85%, 90%, 95%, 99% or 100% sequence identity to SEQ ID NO: 1 and which encodes one or more Rep78, Rep68, Rep52 and Rep40 polypeptides.

In some embodiments, the rep gene is not operably-associated with a functional promoter. In this way, a low level of expression of Rep polypeptides is obtained, wherein the expression level is sufficiently low such as not to prevent adenoviral growth and not to be sufficiently toxic to cells such as to prevent AAV production. In the wild-type AAV, expression of the rep gene products are driven by the p5 and p19 promoters.

As used herein, the term “the rep gene is not operably-associated with a functional promoter” means that the rep gene does not comprise a functional p5 or a functional p19 promoter, and that the rep gene is not operably-associated with any other functional promoter, such that only baseline or minimal transcription of the rep gene is obtained.

In some preferred embodiments of the invention, the transcription of the rep gene will be driven by a polymerase II promoter. The promoter may be inducible or constitutive.

If the promoter is constitutive, then the strength of the promoter should not be too strong such that the rep gene is toxic to the cells.

An adenovirus inhibitor sequence is encoded within the wt AAV rep DNA (located within the p40 promoter that is normally used by the virus for driving expression of the cap genes). Publications have shown that the AAV rep gene can be tolerated within an adenovirus by scrambling this ‘inhibitory’ p40 DNA sequence (Sitaraman, V et al., 2011; Weger, S. et al., J. Virol. 2016).

In some embodiments, the Rep polypeptide encoding sequence does not comprise a functional adenovirus inhibitor sequence. As used herein, the term “functional adenovirus inhibitor sequence” refers to a nucleotide sequence wherein when it is present in cis of the adenovirus genome, it leads to significant inhibition of replication of the adenovirus.

The wild-type AAV2 adenovirus inhibitor has the sequence:

Gtcaccaagcaggaagtcaaagactttttccggtgggcaaaggatcacgt
ggttgaggtggagcatgaattctacgtcaaaaagggtggagccaagaaaa
gacccgcccccagtgacgcagatataagtgagcccaaacgggtgcgcgag
tcagttgcgcagccatcgacgtcagacgcggaagcttcgatcaactacgc
agacaggt (SEQ ID NO: 2)

The above sequence forms the p40 promoter and adenovirus inhibitor sequence. The TATA element (in bold) and transcriptional start site form the core of the inhibitor sequence.

Preferably, a functional adenovirus inhibitor sequence is defined as one which has the sequence shown above, or a variant thereof which has at least 80%, 85%, 90% or 95% sequence identity thereto and which is capable of inhibiting adenoviral vector replication in a host cell.

The level of activity of the adenovirus inhibitor sequence may be determined by including an adenovirus inhibitor sequence (in cis or trans) into the sequence of an AV vector through molecular cloning and then attempting to recover the AV in mammalian cells. The insertion of a wild type adenovirus inhibitor sequence into an AV would completely prevent the recovery and outgrowth of any AV vector. By modifying the sequence of the adenovirus inhibitory sequence, it may be possible to recover AV vectors with varying degrees of success. This can be calculated by measuring the infectious titre of the recovered AV to determine the level of inhibition. Assays that can be used to measure AV titre include the TCID50 method and the plaque assay method.

A level of activity which is less than 5% (preferably less than 1%) of the activity level from a wild-type adenovirus inhibitor sequence (under the same conditions) may be considered to be not functional.

In some preferred embodiments, the adenovirus inhibitor sequence is scrambled, i.e. one or more synonymous mutations are present within p40 cis-inhibitory sequence which ablate its effect in repressing adenovirus replication but still maintain the Rep polypeptide sequence.

In some embodiments of the invention, the recombinant adenovirus comprises a repressor element (e.g. a Tet repressor element) in the Major Late Promoter (MLP).

In some particularly-preferred embodiments, the recombinant adenovirus comprises:
(i) a Tet repressor element in the MLP;
(ii) an AAV rep gene; and optionally
(iii) an AAV cap gene.

WO2019/020992 discloses that transcription of the Late adenoviral genes can be regulated (e.g. inhibited) by the insertion of a repressor element into the Major Late Promoter. By “switching off” expression of the adenoviral Late genes, the cell’s protein-manufacturing capabilities can be diverted toward the production of a desired recombinant protein or AAV particles.

In particular, WO2019/020992 discloses that the adenoviral vector containing a Tet repressor element in the Major Late Promoter can also encode the TetR protein downstream, and under the transcriptional control of the Major Late Promoter. In the absence of doxycycline, the TetR protein will bind to the Major Late Promoter Tet repressor element and prevent the promoter’s activity. In the presence of doxycycline, the TetR protein cannot bind to the Tet repressor element in the Major Late Promoter. Consequently, in the presence of doxycycline, the Major Late Promoter of the adenovirus is active, the structural genes of the adenovirus are expressed and the virus can replicate, and lyse cells.

The full contents of WO2019/020992 are explicitly incorporated herein by reference. In this regard, preferred features include the following:
wherein the MLP comprises one or more repressor elements.
wherein one or more of the repressor elements are inserted downstream of the MLP TATA box.
wherein the one or more repressor elements are inserted between the MLP TATA box and the +1 position of transcription.
wherein the repressor element(s) are capable of being bound by a repressor protein.
wherein a gene encoding a repressor protein which is capable of binding to the repressor element(s) is encoded within the adenoviral genome.
wherein the repressor protein is transcribed under the control of the MLP.
wherein the repressor protein is the tetracycline repressor, the lactose repressor or the ecdysone repressor, preferably the tetracycline repressor (TetR).
wherein the repressor element is a tetracycline repressor binding site, preferably comprising or consisting of the sequence set forth in SEQ ID NO: 3:

tccctatcagtgatagaga

wherein the nucleotide sequence of the MLP comprises or consists of the sequence set forth in SEQ ID NO: 4 or 5.

An example of a modified MLP which contains one TetR binding site between the TATA box and the +1 position of transcription is given below:

Cgccctcttcggcatcaaggaaggtgattggtttgtaggtgtaggccacg
tgaccgggtgttcctgaaggggggctataaaaggtccctatcagtgatag
agactca (SEQ ID NO: 4)

The TATA box is underlined and the TetR binding site is shown in bold.

An example of a modified MLP which contains one TetR binding site between the TATA box and the +1 position of transcription and a second site upstream of the TATA box between the UPE element and the TATA box is shown below:

Cgccctcttcggcatcaaggaaggtgattggtttgtaggtgtaggccacg
tgactccctatcagtgatagagaactataaaaggtccctatcagtgatag
agactca (SEQ ID NO: 5)

The TATA box is underlined and the TetR binding site is shown in bold.

Other preferred features include the following:

wherein the presence of the repressor element does not affect production of the adenoviral E2B protein.

It is particularly preferred that one or more of the repressor elements are inserted downstream of the MLP TATA box.

Step (a) also comprises the step of providing (e.g. dispensing) a solution having a defined level of dilution of the sample of wild type or recombinant AAVs, into one or each of the set of discrete compartments or subsets of discrete compartments. The AAVs must be capable of infecting the host cells.

As used herein, the term “recombinant adeno-associated virus” or rAAV refers to an AAV comprising 5’- and 3’-AAV inverted terminal repeats (ITRs), wherein one or more heterologous genes have been inserted, the rep gene has been modified (to make it non-functional) or deleted, and one or more other non-natural modifications have been made. The rAAV does not therefore have a wild-type AAV nucleotide sequence. The rAAV may encode a cap gene. Preferably, the rep has been deleted from the rAAV.

In some embodiments, the rep gene (and optionally also the cap gene) has been deleted, and a transgene has been inserted.

In some embodiments, the AAV serotype is preferably selected from the group consisting of serotypes 1-9, AAVrh10, AAVrh32.33, AAV-DJ, AAV-DJ8, AAV-PHP.B and AAV-PHP.eB.

A solution having a defined level of dilution of the sample of wild-type or recombinant AAVs is provided or dispensed into each of the one or set of discrete compartments.

The sample (i.e. parent population of AAVs whose titre is being determined) will generally be in the form of a liquid (e.g. aqueous) composition. The solution may be a dilution media. The dilution media may be any media which is compatible with the method of the invention. The dilution media may, for example, be the culture media in which the host cells are cultured. Each of the discrete compartments in the set of discrete compartments may receive a different level of dilution of the sample of AAV particles.

Different subsets of compartments within the set of discrete compartments may receive different defined levels of dilution of the population of rAAVs. The subsets of compartments may, for example, be specific rows or columns of a multi-well plate.

The set of discrete compartments will comprise at least 2 subsets, preferably 3, 4, 5, 6, 7, 8, 9, 10, 11, 12, 13, 14, 15 or more subsets. For example, a first subset of compartments (e.g. rows or columns of a micro-titre plate) may receive a first defined level of dilution of the population of AAVs, wherein there are n subsets of compartments, and wherein the nth subset of compartments receives a d⁻⁽ⁿ⁻¹⁾ dilution of the first defined level of dilution of the population of AAVs, where d is the serial dilution factor, wherein n=2 to 20. Preferably, d = 10.

For example, the first dilution of the parent population of rAAVs may be a 10⁻¹, 10⁻², 10⁻³, 10⁻⁴, 10⁻⁵, 10⁻⁶, 10⁻⁷ or 10⁻⁸ dilution. In some preferred embodiments, the first dilution is a 10-⁴ dilution.

Sets of second and further dilutions may then be made, wherein the second and further dilutions are dilutions of the preceding dilution.

The dilution ratio between dilutions may be any defined ratio, which may or may not be the same between each pair of dilutions; preferably it is the same ratio. Preferably, the dilution ratio between each pair of subsequent dilutions is 1:10 or 1:100, more preferably 1:10.

In this way, a set of dilutions may be made. For example, the set of dilutions may comprise the following:

| | |
|---|---|
| First dilution | 1:10⁴ of the parent population of AAVs |
| Second dilution | 1:10⁵ of the parent population of AAVs |
| Third dilution | 1:10⁶ of the parent population of AAVs |
| Fourth dilution | 1:10⁷ of the parent population of AAVs |
| Fifth dilution ... etc. up to | 1:10⁸ of the parent population of AAVs |
| Ninth dilution | 1:10¹² of the parent population of AAVs |

Each of the dilutions may be produced more than once, e.g. in duplicate or triplicate.

Components (i)-(iii) may be dispensed into the one or set of discrete compartments in any order. Preferably, the host cells are dispensed into the one or set of discrete compartments first, and cultured therein until the host cells are adequately confluent. The recombinant adenoviruses may then be added at an appropriate MOI, followed by the diluted sample of wild-type or recombinant AAVs.

Step (b) comprises:

(b) culturing components (i)-(iii) in the set of discrete compartments under conditions such that the host cells are infected by the recombinant adenoviruses and the wild-type or recombinant AAVs.

The components are cultured for a time which allows for infection of the host cells by the recombinant adenoviruses and the wild-type or recombinant AAVs; and replication of the AAVs. Such conditions are well known in the art. One example is incubation at 37° C., 5% CO₂, 85% humidity, for 2 days.

The cells are cultured in a culture medium, e.g. a liquid culture medium. Any suitable culture media may be used, e.g. DMEM High Glucose (Sigma-Aldrich), supplemented with 10% FBS (Gibco Life Technologies).

Step (c) comprises:

(c) determining the level of a biomarker for each of the discrete compartments, wherein the biomarker is one which is representative of the number of wild-type or recombinant AAV particles, and thereby determining the titre of the AAV particles in the sample.

The level of the biomarker may be detected either directly or indirectly. The detection method may vary depending on the nature of the biomarker moiety (e.g. AAV gene) or effect (e.g. fluorescence) being detected. Examples of detection methods include fluorescence microscopy, automated fluorescence cell imaging and quantification of cellular DNA extracted by biomarker-specific (e.g. EGFP) qPCR.

The biomarker is one which is representative of the number of AAVs, i.e. the level of the biomarker in any one of the discrete compartments is representative of the number of wild-type or recombinant AAVs in that discrete compartment.

As used herein, the term “representative” means that the level of the biomarker in any one of the discrete compartments is directly proportional to the number of wild-type or recombinant AAVs in that discrete compartment.

For example, the biomarker may be a gene or other nucleotide sequence which is present in the AAV genome, or a polypeptide which is encoded by a gene which is present in the AAV genome.

Examples of biomarker genes include a reporter gene or transgene which is present in the genome of the recombinant AAV; or a rep gene or cap gene which is present in a wild-type AAV. The presence of and levels of such genes may be determined by qPCR, PCR or Southern blotting.

Other examples of nucleotide sequences which may be used as biomarkers include a transgene promoter (e.g. CMV), a poly-adenylation elements (e.g. SV40 polyA, BgH polyA) and AAV inverted terminal repeat (ITRs). Such nucleotide sequences may be present in wild-type or recombinant AAVs.

Examples of biomarker polypeptides include the expression products of reporter genes or transgenes which are present in the genome of the recombinant AAV. The presence of and levels of such polypeptides may be determined by detection of fluorescence, luminescence or colour change.

For example, the presence of and levels of EGFP, mCherry, DsRed, YFP, and RFP may detected in rAAV by fluorescence detection. The presence of and levels of Luciferase and SEAP may be detected in rAAV by luminescence detection. The presence of and levels of β-galactosidase in rAAV may be detected by colometric assay. The presence of and levels of hFIX in rAAV may be detected by enzyme activity.

Other examples of biomarker polypeptides include therapeutic polypeptides which are encoded by transgenes which are present in the genome of the recombinant AAV, such as human Factor IX. The presence of and levels of such polypeptides may be determined by antibody staining or by enzymatic activity.

Preferably, the biomarker is all or a detectable part of the AAV genome, and the detection method is by qPCR.

The level of the biomarker may be determined from the culture medium or from the AAV particles, as appropriate, depending on the biomarker to be determined. The level of the biomarker in each of the discrete compartments may be determined after a defined time period. Examples of suitable time periods include 1, 2, 3 or 4 days, preferably after 2 days.

The titre of recombinant or wild-type AAVs in the sample may then readily be determined by using knowledge of the level of biomarker found in a particular discrete compartment, the level of sample dilution which was provided in that discrete compartment, and a standard or control assay correlating known levels of biomarkers with known numbers of AAVs.

In some embodiments, step (c) comprises the steps of:
(c)(i) determining the level of a biomarker for each of the discrete compartments, wherein the biomarker is one which is representative of the number of AAVs; and
(c)(ii) comparing the determined levels of biomarkers for one or more of the discrete compartments against the levels obtained from a standard or control assay correlating known levels of biomarkers with known numbers of AAVs;
and thereby determining the titre of the AAVs in the sample.

TCID₅₀ (Median Tissue Culture Infectious Dose) is one of the methods used when verifying viral titre. The TCID₅₀ is the concentration at which 50% of the cells are infected when a test tube or well plate upon which cells have been cultured is inoculated with a diluted solution of viral fluid.

TCID₅₀ per mL may be calculated using the KÄRBER-SPEARMAN statistical method (Cawood, R., et al. “Use of tissue-specific microRNA to control pathology of wild-type adenovirus without attenuation of its ability to kill cancer cells”. PLoS Pathog 5, e1000440 (2009)), as given in the formula below:

$$T i t r e \left(T C I D 50 / m L\right) = \frac{{10}^{1 + D \left({X}_{0} + S - 0.5\right)}}{v o l u m e p e r w e l l \left[m L\right]}$$

where D = log(d); d = serial dilution factor (e.g. when d=10, D=log10=1); X₀ = -log [the highest dilution factor with which all wells in the row in a micro-titre plate are positive] (e.g. if at 1:10⁵, 1:10⁶ and 1:10⁷ dilutions, all wells in the rows are positive, while at 1:10⁸ and further dilutions fewer than all of the wells are positive in the rows, then X₀ = 7 in this case).

S = sum of the ratio of positive wells in a row (or more than one row) where not all wells are positive (e.g. a row with 8/10 positive wells means the ratio of positive wells is 0.8 for this row; but if all wells in a row are all positive, this should not be counted when calculating S).

In yet a further embodiment, therefore, the invention provides a method of determining the TCID₅₀ (Median Tissue Culture Infectious Dose) of a population of rAAVs, the method comprising the steps:
(a) performing a method of the invention, wherein different subsets of compartments (e.g. rows in a micro-titre plate) receive different defined levels of the population of AAVs and the different defined levels vary by the serial dilution factor, d; and
(b) calculating the TCID₅₀ using the formula:
$$T i t r e \left(T C I D 50 / m L\right) = \frac{{10}^{1 + D \left({X}_{0} + S - 0.5\right)}}{v o l u m e p e r c o m p a r t m e n t \left[m L\right]}$$
wherein
$$\text{D=log} \left(\text{d}\right) ;$$
X₀ = -log [the highest dilution factor at which the presence of rAAVs has been detected in all compartments (e.g. wells) in a particular subset of compartments (e.g. rows in a micro-titre plate)]; and
wherein, in the subsets of compartments (e.g. rows of a micro-titre plate) in which the presence of rAAVs has not been detected in all compartments (e.g. wells of the micro-titre plate) in that subset, S is the sum of the ratios of the number of compartments (e.g. wells) in which rAAVs have been detected to the total number of compartments (e.g. wells) in that subset of compartments (e.g. rows). Preferably, serial dilution factor, d = 10.

There are many established algorithms available to align two amino acid or nucleic acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid or nucleic acid sequences for comparison may be conducted, for example, by computer-implemented algorithms (e.g. GAP, BESTFIT, FASTA or TFASTA), or BLAST and BLAST 2.0 algorithms.

Percentage amino acid sequence identities and nucleotide sequence identities may be obtained using the BLAST methods of alignment (Altschul et al. (1997), “Gapped BLAST and PSI-BLAST: a new generation of protein database search programs”, Nucleic Acids Res. 25:3389-3402; and http://www.ncbi.nlm.nih.gov/BLAST). Preferably the standard or default alignment parameters are used.

Standard protein-protein BLAST (blastp) may be used for finding similar sequences in protein databases. Like other BLAST programs, blastp is designed to find local regions of similarity. When sequence similarity spans the whole sequence, blastp will also report a global alignment, which is the preferred result for protein identification purposes. Preferably the standard or default alignment parameters are used. In some instances, the “low complexity filter” may be taken off.

BLAST protein searches may also be performed with the BLASTX program, score=50, wordlength=3. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. (See Altschul et al. (1997) supra). When utilizing BLAST, Gapped BLAST, PSI-BLAST, the default parameters of the respective programs may be used.

With regard to nucleotide sequence comparisons, MEGABLAST, discontiguous-megablast, and blastn may be used to accomplish this goal. Preferably the standard or default alignment parameters are used. MEGABLAST is specifically designed to efficiently find long alignments between very similar sequences. Discontiguous MEGABLAST may be used to find nucleotide sequences which are similar, but not identical, to the nucleic acids of the invention.

The BLAST nucleotide algorithm finds similar sequences by breaking the query into short subsequences called words. The program identifies the exact matches to the query words first (word hits). The BLAST program then extends these word hits in multiple steps to generate the final gapped alignments. In some embodiments, the BLAST nucleotide searches can be performed with the BLASTN program, score=100, wordlength=12.

One of the important parameters governing the sensitivity of BLAST searches is the word size. The most important reason that blastn is more sensitive than MEGABLAST is that it uses a shorter default word size (11). Because of this, blastn is better than MEGABLAST at finding alignments to related nucleotide sequences from other organisms. The word size is adjustable in blastn and can be reduced from the default value to a minimum of 7 to increase search sensitivity.

A more sensitive search can be achieved by using the newly-introduced discontiguous megablast page (www.ncbi.nlm.nih.gov/Web/Newsltr/FallWinter02/blastlab.html). This page uses an algorithm which is similar to that reported by Ma et al. (Bioinformatics. 2002 Mar; 18(3): 440-5). Rather than requiring exact word matches as seeds for alignment extension, discontiguous megablast uses non-contiguous word within a longer window of template. In coding mode, the third base wobbling is taken into consideration by focusing on finding matches at the first and second codon positions while ignoring the mismatches in the third position. Searching in discontiguous MEGABLAST using the same word size is more sensitive and efficient than standard blastn using the same word size. Parameters unique for discontiguous megablast are: word size: 11 or 12; template: 16, 18, or 21; template type: coding (0), non-coding (1), or both (2).

In some embodiments, the BLASTP 2.5.0+ algorithm may be used (such as that available from the NCBI) using the default parameters.

In other embodiments, a BLAST Global Alignment program may be used (such as that available from the NCBI) using a Needleman-Wunsch alignment of two protein sequences with the gap costs: Existence 11 and Extension 1.

As used herein, the term “sequence identity” in the context of amino acid sequences may alternatively be replaced by “sequence similarity”. The term “similarity” allows conservative substitutions of amino acid residues having similar physicochemical properties over a defined length of a given alignment. The percentage of similarity is determinable with any reasonable similarity-scoring matrix.

The DNA molecules, plasmids and vectors of the invention may be made by any suitable technique. Recombinant methods for the production of the nucleic acid molecules and packaging cells of the invention are well known in the art (e.g. “Molecular Cloning: A Laboratory Manual” (Fourth Edition), Green, MR and Sambrook, J., (updated 2014)).

Preferably, the method steps are carried out in the order specified.

The disclosure of each reference set forth herein is specifically incorporated herein by reference in its entirety. In particular, the definitions of the TERA and TESSA vectors as described in WO2021/156609 are specifically incorporated herein by reference.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: TCID50 of Recombinant AAV Comprising an EGFP Reporter Gene

### Seeding, Day 0

1. Seed HEK293 cells in 96-well tissue culture plate(s) at 1.5 × 10⁵ viable cells/mL (100 µL volume per well).

Note: A full 96 well assay plate is required for each rAAV-EGFP sample to be assayed. Each assay plate consists of 10 replicate wells of 8 concentrations of the rAAV-EGFP sample.

2. Place culture plate(s) into the static incubator (37° C., 5% CO₂ and 85% humidity) for 16-24 hours before infection.

### Infection, Day 1

3. Approximately 24 hours post cell seeding, check the confluency of the cells using a light microscope. For optimal infection, the cells should be ~90% confluent.

4. Establish cell count using wells H11 and H12 from each 96-well assay plate. Each well should contain approximately 2.0 × 10⁴ cells per well.

5. Thaw recombinant adenovirus (TESSA-RepCap2) vectors at 37° C. and use at room temperature.

The vector “TESSA-RepCap2” encodes AAV2 Rep and Cap. In this construct, there are no functional Rep p5 or p40 promoters; no Rep adenovirus inhibitor sequence is present; and the Rep p19 promoter has been modified to delete the TATA box, although the promoter is still functional. The rep gene is inserted in the E1 region of the adenovirus. Cap is expressed from a CMV promoter. The cap gene is inserted into the E1 region of the adenovirus. The transcriptional orientation of the CMV promoter does not drive towards the Rep coding sequence. The cap gene is from AAV2.

6. For each 96-well assay plate, you will need to prepare 15 mL infection media, calculating the amount of TESSA-RepCap2 required using the formula below. TESSA-RepCap2 is used for infection at an MOI of 15 TCID50 per cell.

$$\text{Volume} \text{of} \text{TESSA-RepCap2} \left(μ \text{L}\right) = \left(\left(\text{cells} \text{per} \text{well}\right) / \text{x}\right) ∗ \left(15 )\right) ∗ 150$$

where x is the TCID50/µL of TESSA-RepCap2, ‘15’ is the TCID50 per cell and ‘150’ is the excess number of wells required for each 96-well plate.

Make up the volume of TESSA-RepCap2 (µL) to a total volume of 15 mL with DMEM High Glucose supplemented with 2% FBS (45 mL for a triplicate assay).

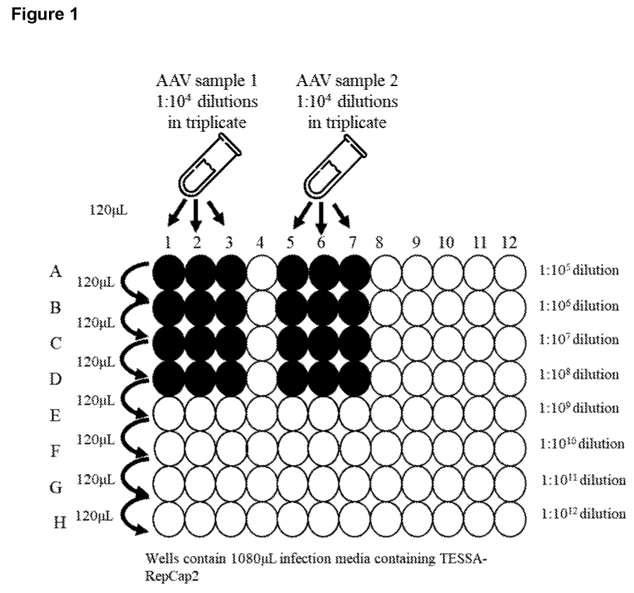

7. Aliquot 1080 µL of the infection media into each well of column 1 of a sterile 96 Deep Well Plate (DWP), “the dilution plate” (see FIG. 1). For a triplicate assay or multiple samples for titration, repeat for columns 2 and 3, etc.

8. Prepare a 1:10,000 dilution of the rAAV-EGFP to be analysed as follows:
a. 1:100 dilution: Add 10 µL of viral sample to 990 µL infection media in a 1.5 mL microfuge tube to achieve a 1:100 dilution.
b. 1:10,000 dilution: Add 10 µL of 1:100 diluted viral sample to 990 µL infection media in a 1.5 mL microfuge tube to achieve a 1:10,000 dilution.

9. Add 120 µL of the 1:10,000 dilution of rAAV-EGFP into well A1 of the dilution plate containing 1080 µL of the infection media prepared in step 7. Gently pipette up and down 20 times to mix.

10. Prepare 10-fold serial dilutions of the sample in row A by transferring 120 µL of infection media/sample mixture from row A into the corresponding well of row B. Gently pipette up and down 20 times to mix.

11. Repeat this for every row down the dilution plate to reach the highest dilution (1 ×10-12 in row H).

12. Using a multi-channel pipette, transfer 100 µL of the infection media/rAAV-EGFP mix from the 96 DWP to the 96-well assay plate containing cells prepared in step 1, as follows. One column in the 96 DWP (dilution plate) requires 1×96-well assay plate, and for triplicate assays and/or multiple samples, more assay plates are required accordingly.
a. 1:10⁵ dilution (DWP Row A well) to 96 well plate wells A1 -A10
b. 1:10⁶ dilution (DWP Row B well) to 96 well plate wells B1-B10
c. 1:10⁷ dilution (DWP Row C well) to 96 well plate wells C1-C10
d. Repeat until row H (1:10¹² dilution)

13. Add 100 µL of infection media, not containing rAAV-EGFP, to each well in columns 11-12 of the 96-well plate as a negative control.

14. Transfer the 96-well plate to the static incubator and incubate for 2 days using the same incubator setting as in step 2.

### Titration Analysis, Day3

15. Inspect the 96 well plate with a fluorescent microscope using a filter set optimised for EGFP (488 nm excitation). Note down each well that is positive; a well is positive when one or more cells were observed to express EGFP. Negative control wells should not contain EGFP expressing cells.

16. Acceptance criteria: all wells at the lowest dilutions (A1-10) must be positive and all wells at the highest dilution (H1-10) are negative. If the assay does not meet the acceptance criteria, the dilution range should be adjusted to accommodate the titre range of rAAV stocks.

17. TCID₅₀ per mL is calculated using the KÄRBER-SPEARMAN statistical method (formula below):

$$T i t r e \left(T C I D 50 / m L\right) = \frac{{10}^{1 + D \left(X 0 + S - 0.5\right)}}{v o l u m e p e r w e l l \left[m L\right]}$$

Where D = log(d); d = serial dilution factor (i.e. d=10 in the method described here, so D=log10=1);

X₀ = -log (the highest dilution factor with which all wells in the row are EGFP-positive) (e.g. if at 1:10⁵, 1:10⁶ and 1:10⁷ dilutions, all 10 wells in the rows are positive, while at 1:10⁸ and further dilutions fewer than 10 wells are positive in the rows, then X₀ = 7 in this case)

S = sum of the ratio of positive wells in a row where not all wells are EGFP-positive (e.g. a row with 8 EGFP-positive wells means the ratio of positive wells is 0.8 for this row; but if 10 wells in a row are all positive, this should not be counted when calculating S).

Volume per well is 0.1 mL as described in this protocol.

### Example 2: Infectivity of AAV2-EGFP Stocks Quantified using Adenovirus Vector TERA Encoding AAV Rep and Cap2 Genes

AAV2-EGFP vectors were produced in HEK293 cells using the TERA2.0 (TERA-AAV-EGFP and TERA-RepCap2 are as described in WO2021/156609) or by transfection with the helper-free plasmids (pAAV-EGFP, pRepCap2 and pHelper). Crude AAV2-EGFP preparations were quantified by the TCID50 assay in HEK293 cells, with and without the addition of TERA-RepCap2. In the TESSA-RepCap2 group, cells were observed for EGFP expression at day 3 post infection, while EGFP expression was recorded at day 7 in samples without TESSA-RepCap2 co-infection.

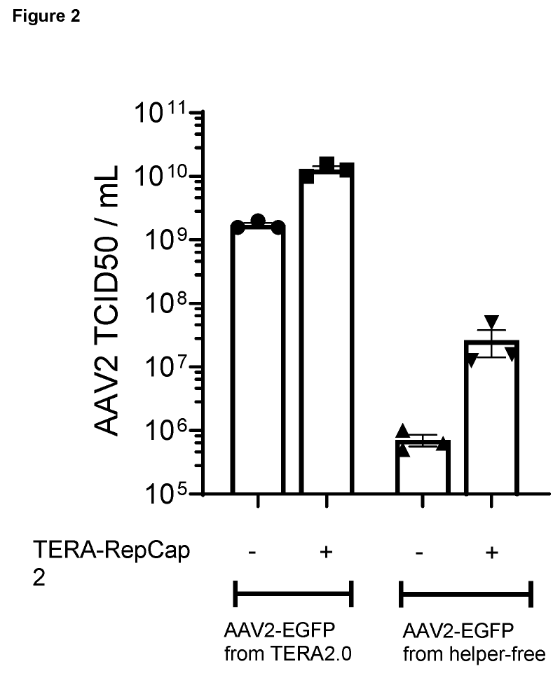

The results shown in FIG. 2 demonstrate that the addition of TESSA-RepCap2 enables the sensitive detection of cells transduced with AAV2-EGFP.

### Example 3: DNA Replication of rAAV2-EGFP Vectors in A549 Cells Using TERA-E1-Rep Virus, Wherein the Rep Gene is Integrated Alongside the E1 Genes within the Recombinant Adenovirus.

rAAV2-EGFP vectors produced in HEK293 cells by transfection with the helper-free plasmids (pAAV-EGFP, pRepCap2 and pHelper) were used to infect A549 cells at 100 genome copies per cell alongside TERA-E1-Rep or control adenoviruses TERA-E1 (without AAV Rep), Ad5-E1 (without AAV Rep), TERA-Rep (without Ad5 E1 genes) used at 100 genome copies per cell. Total DNA were extracted from infected cells at 24-, 48-, 72- and 96-hours post-infection and quantified by EGFP-specific qPCR.

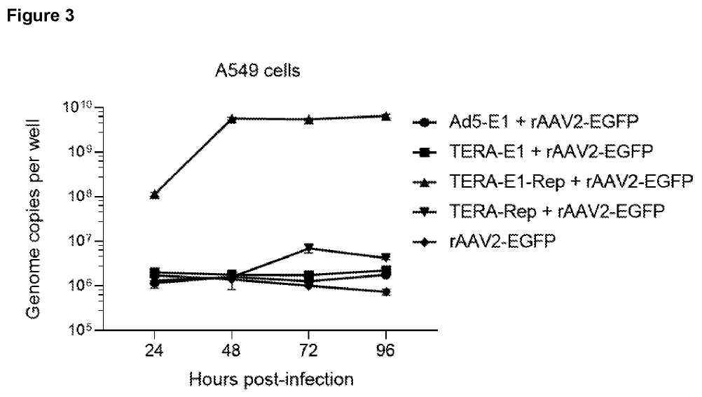

The results shown in FIG. 3 demonstrate that the TERA-E1-Rep adenovirus enables significant replication of rAAV-EGFP genomes within infected A549 cells that lack the adenovirus E1 genes.

### Example 4: Detection of rAAV2-EGFP Infection Event in HEK293 (TESSA-E1-Rep Aided) and HelaRC32 (Ad-E1 Aided) Cells

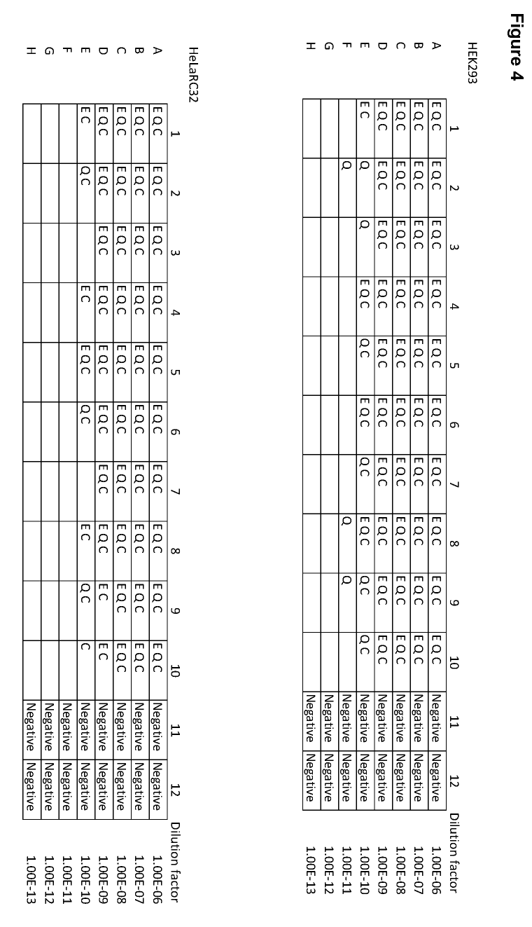

Detection of rAAV2-EGFP infection event in HEK293 cells was tested using the TERA-E1-Rep virus and compared to the HeLaRC32 cells titration method via co-infection with Ad5-E1 virus. rAAV2-EGFP were produced in HEK293 cells using the TERA2.0 method (TERA-AAV-EGFP and TERA-RepCap2, as described in WO2021/156609). HEK293 and HeLaRC32 cells were seeded in 96-well tissue culture plates at a density of 1e4 cells and 2e4 per well, respectively, for 24 hours. Eight 10-fold serial dilutions of purified rAAV2-EGFP stock were made in DMEM containing 2% FBS (supplemented with TERA-E1-Rep for HEK293, and Ad5-E1 for HeLaRC32 cells, at an MOI of 10) for at a total volume of 1.2 mL. Ten replicates of each diluted sample (1e10-6 to 1e10-13) were added at a volume of 100 µL per well on each plate. At day 3 after infection, each well was examined for rAAV2-EGFP infection event via fluorescence microscopy (E), automated Fluorescence cell imager (C), or cellular DNA extracted from each well and quantified by EGFP-specific qPCR (Q). The results are shown in FIG. 4. This example shows that the rAAV2-EGFP infection events in HEK293 cells (infected with TERA-E1-Rep) can be robustly detected via qPCR and EGFP expression.

### Example 5: Infectious Titration of AAV2-EGFP in HEK293 Cells and HeLaRC32 Cells using qPCR, EVOS or Cell Imager Detection

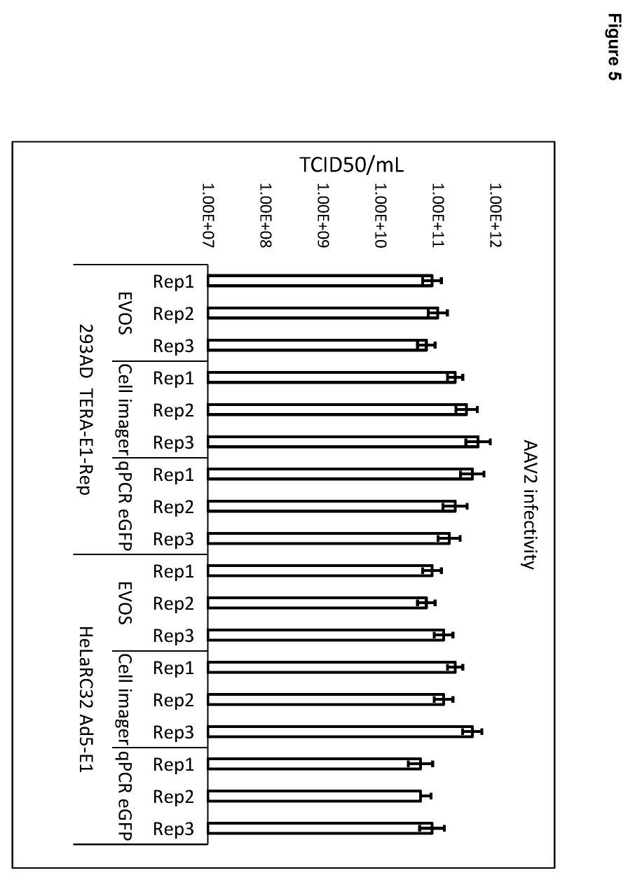

Infectious titration of rAAV2-EGFP stock in HEK293 and HeLaRC32 cells was assayed using the TCID50 assay. HEK293 and HeLaRC32 cells were seeded in 96-well tissue culture plates. Eight 10-fold serial dilutions of purified rAAV stocks were made in DMEM containing 2% FBS (supplemented with TERA-E1-Rep at an MOI of 10) for a total volume of 1.2 mL. Ten replicates of each diluted sample (1 e10-6 to 1e10-13) were added at a volume of 100 µL per well on each plate in a TCID50 assay (three replicate plates, Rep1-3). At Day 3 after infection, wells in each plate were examined for EGFP expression using a fluorescence microscope (EVOS), automated fluorescence cell imager (Cell imager; CELLAVISTA) or total DNA was extracted from each well and measured by EGFP-specific qPCR. Infectious rAAVs were determined as TCID50 per mL using the KÄRBER formula. The results are shown in FIG. 5. This example shows the infectious titration of rAAV2-EGFP in HEK293 cells with TERA-E1-Rep using the EGFP reporter or qPCR detection.

### Example 6: Infectious Titration of rAAV2 in Different Cell Lines With TERA-E1-Rep Compared to HeLaRC32 Method using the TCID50 Assay

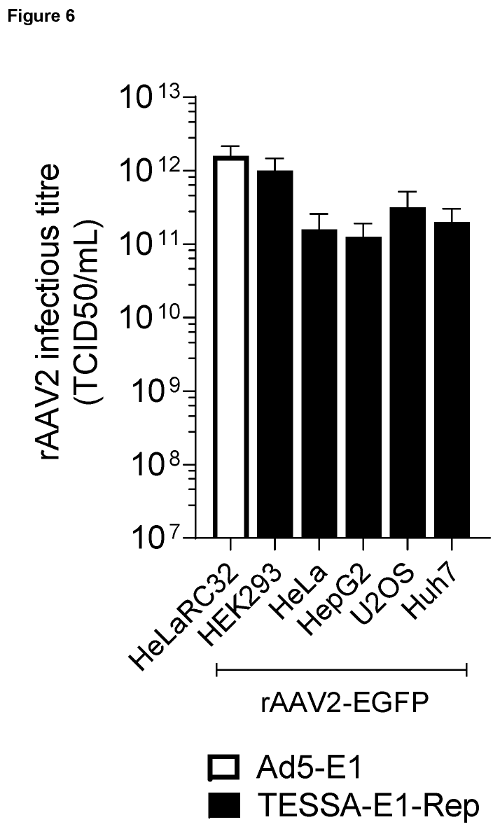

Infection titration of rAAV-EGFP in HEK293, HeLa, HepG2, U2OS and Huh7 cells with TERA-E1-Rep using the TCID50 assay compared to infectious titration using HeLa RC32 co-infected with Ad5-E1. rAAV2-EGFP were produced in HEK293 cells using the TERA2.0 method (TERA-AAV-EGFP and TERA-RepCap2 as described in WO2021/156609). Cells were seeded in 96-well tissue culture plates. Eight 10-fold serial dilutions of purified rAAV2-EGFP stock were made in DMEM containing 2% FBS (supplemented with TERA-E1-Rep for HEK293, HeLa, HepG2, U2OS and Huh7, or Ad5-E1 for HeLa RC32 cells, at an MOI of 10) for a total volume of 1.2 mL. Ten replicates of each diluted sample (1e10-6 to 1e10-13) were added at a volume of 100 µL per well on each plate. At day 3 after infection, total DNA was extracted from each well, and positive infectious events were determined by EGFP-specific qPCR. Infectious rAAVs were determined as TCID50 per mL. The results are shown in FIG. 6. This example shows the infectious titer of the rAAV-EGFP stock can be determined in the various cell lines using the TERA-E1-Rep virus.

### Example 7: Genome Copies to TCID50 Ratio of AAV2-EGFP Stock in Different Cell Lines

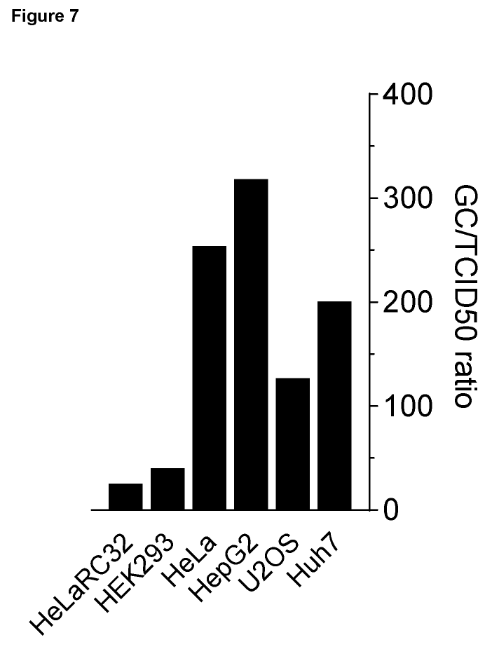

Infection titration of rAAV-EGFP in HEK293, HeLa, HepG2, U2OS and Huh7 cells with TERA-E1-Rep using the TCID50 assay were compared to infectious titration using HeLa RC32 co-infected with Ad5-E1. rAAV2-EGFP were produced in HEK293 cells using the TERA2.0 method (TERA-AAV-EGFP and TERA-RepCap2 as described in WO2021/156609). Cells were seeded in 96-well tissue culture plates. Eight 10-fold serial dilutions of purified rAAV2-EGFP stock were made in DMEM containing 2% FBS (supplemented with TERA-E1-Rep for HEK293, HeLa, HepG2, U2OS and Huh7, or Ad5-E1 for HeLa RC32 cells, at an MOI of 10) for a total volume of 1.2 mL. Ten replicates of each diluted sample (1e10-6 to 1e10-13) were added at a volume of 100 µL per well on each plate. At day 3 after infection, total DNA was extracted from each well, and positive infectious events were determined by EGFP-specific qPCR. Infectious rAAVs were determined as TCID50 per mL using the KÄRBER-SPEARMAN formula and compared against genome copies (GC) of rAAV2-EGFP vector measured using qPCR. The results are shown in FIG. 7. This example shows the GC/TCID50 ratio of the rAAV-EGFP stock can be determined in the various cell lines using the TERA-E1-Rep virus by comparing it against the qPCR titer of the AAV2-EGFP stock.

### Example 8: Infectious Titre of rAAV Serotypes Determined in HEK293 and A549 Cells With TERA-E1-Rep using the TCID50 Assay

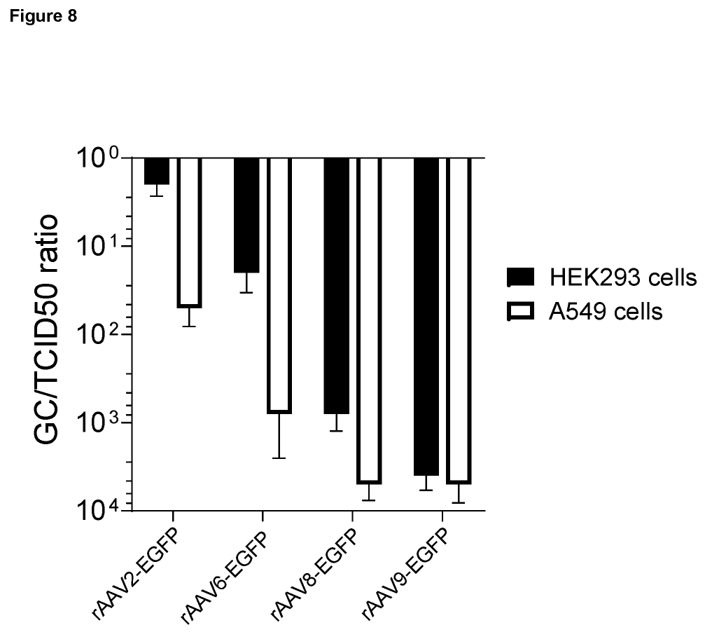

Infectious titre of rAAV2-EGFP, rAAV6-EGFP, rAAV8-EGFP, and rAAV9-EGFP stocks were determined in HEK293 and A549 cells using TERA-E1-Rep. HEK293 and A549 cells were seeded in 96-well tissue culture plates. Eight 10-fold serial dilutions of purified rAAV stocks were made in DMEM containing 2% FBS (supplemented with TERA-E1-Rep at an MOI of 10) for a total volume of 1.2 mL. Ten replicates of each diluted sample (1e10-5 to 1e10-12) were added at a volume of 100 µL per well on each plate in a TCID50 assay. At day 3 after infection, total DNA was extracted from each well, and positive infectious events were determined by EGFP-specific qPCR. Infectious rAAVs were determined as TCID50 per mL using the KÄRBER-SPEARMAN formula and compared against genome copies (GC) of rAAV-EGFP stocks measured using qPCR. The results are shown in FIG. 8. This example shows that the GC/TCID50 ratio of different rAAV-EGFP serotypes can be determined in both HEK293 and A549 cells using the TERA-E1-Rep virus in a TCID50 assay.

1. A method of determining the titre of recombinant adeno-associated viruses (rAAVs) in a sample of rAAVs, the method comprising the steps of:
(a) providing each of components (i)-(iii) in one or each of a set of discrete compartments:
(i) a population of host cells;
(ii) a population of recombinant adenoviruses, wherein the genome of each recombinant adenovirus comprises a rep gene; and
(iii) a solution having a defined level of dilution of the sample of rAAVs,
wherein the host cells are ones which are capable of being infected by the recombinant adenoviruses and by the rAAVs;
(b) culturing the discrete compartments under conditions such that the host cells are infected by the recombinant adenoviruses and rAAVs; and
(c) determining the level of a biomarker for each of the discrete compartments, wherein the biomarker is one which is representative of the number of rAAVs, and thereby determining the titre of the rAAVs in the sample.

2. The method as claimed in claim 1, wherein the discrete compartments are wells in a multi-well plate or micro-titre plate.

3. The method as claimed in claim 1, wherein the cells are ones which do not have one or more adenoviral Early genes stably integrated into the cell genome or present in an episome within the cell.

4. The method as claimed in claim 1, wherein the recombinant adenovirus has functional E1A and E1B genes.

5. The method as claimed in claim 1, wherein the rep gene in the recombinant adenovirus is not operably-associated with a functional promoter.

6. The method as claimed in claim 1, wherein the rAAV genome comprises a scrambled p40 cis-inhibitory sequence.

7. The method as claimed in claim 1, wherein the genome of each recombinant adenovirus additionally comprises a repressor element in the Major Late Promoter (MLP).

8. The method as claimed in claim 7, wherein one or more of the repressor elements are inserted downstream of the MLP TATA box.

9. The method as claimed in claim 7, wherein one or more of the repressor elements is a tetracycline repressor binding site.

10. The method as claimed in claim 1, wherein the genome of the rAAV does not comprise a rep gene.

11. The method as claimed in claim 1, wherein the biomarker is a gene or other nucleotide sequence which is present in the genome of the rAAV, or a polypeptide which is encoded by a gene which is present in the genome of the rAAV.

12. The method as claimed in claim 1 wherein, in Step (a), component (iii) is provided into different subsets of compartments within the set of discrete compartments, wherein different subsets of compartments receive different defined levels of dilution of the sample of AAVs, and Step (c) comprises (c) determining the level of a biomarker for each of the subsets of discrete compartments, wherein the biomarker is one which is representative of the number of AAVs, and thereby determining the titre of the AAVs in the sample.

13. The method as claimed in claim 12 wherein, in Step (a), a first subset of compartments receive a first defined level of dilution of the sample of AAVs, there are n subsets of compartments, and the nth subset of compartments receives a 10⁻⁽ⁿ⁻¹⁾ dilution of the first defined level of dilution of the sample of AAVs, wherein n=2 to 20.

14. A method of determining the TCID₅₀ (Median Tissue Culture Infectious Dose) of a population of recombinant AAVs, the method comprising the steps:
(a) performing a method as claimed in claim 1,
wherein different subsets of compartments (e.g. rows in a micro-titre plate) receive different defined levels of the sample of rAAVs and the different defined levels vary by the serial dilution factor, d; and
(b) calculating the TCID₅₀ using the formula:
$$T i t r e \left(T C I D 50 / m L\right) = \frac{{10}^{1 + D \left(X 0 + S - 0.5\right)}}{v o l u m e p e r c o m p a r t m e n t \left[m L\right]}$$
wherein
D = log(d);
X₀ = -log [the highest dilution factor at which the presence of rAAVs has been detected in all compartments (e.g. wells) in a particular subset of compartments; and
wherein, in the subsets of compartments (e.g. rows of a micro-titre plate) in which the presence of rAAVs has not been detected in all compartments in that subset, S is the sum of the ratios of the number of compartments in which AAVs have been detected to the total number of compartments in that subset of compartments.

15. A method of determining the titre of wild-type adeno-associated viruses (AAVs) in a sample of AAVs, the method comprising the steps of:
(a) providing each of components (i)-(iii) in one or each of a set of discrete compartments:
(i) a population of host cells;
(ii) a population of recombinant adenoviruses, wherein the genome of each recombinant adenovirus comprises a repressor element in the Major Late Promoter (MLP); and
(iii) a solution having a defined level of dilution of the sample of AAVs,
wherein the host cells are ones which are capable of being infected by the recombinant adenoviruses and by the AAVs;
(b) culturing the discrete compartments under conditions such that the host cells are infected by the recombinant adenoviruses and AAVs; and
(c) determining the level of a biomarker for each of the discrete compartments, wherein the biomarker is one which is representative of the number of AAVs, and thereby determining the titre of the AAVs in the sample.
